(19) United States
(12) Patent Application Publication
(10) Pub. No.: US 2006/0115502 A1
(43) Pub. Date: Jun. 1, 2006

(54) Cosmetic composition based on cirsimarin

(75) Inventors: Alain Geloen, Lyon (FR); Frederic Demarne, Marseille (FR); Catherine Girotti, Lyon (FR)

Correspondence Address: HESLIN ROTHENBERG FARLEY & MESITI PC, 5 COLUMBIA CIRCLE, ALBANY, NY, 12203, US

(73) Assignee: GATTEFOSSE SAS, Saint Priest (FR)

(21) Appl. No.: 10/541,646

(22) PCT Filed: Dec. 30, 2003
(86) PCT No.: PCT/FR03/03938

§ 371 Date: Jul. 6, 2005

(30) Foreign Application Priority Data
Jan. 9, 2003 (FR) 0300197

(51) Int. Cl. A61K 8/49; A61K 31/7048; A61K 31/522; A61K 31/353

(52) U.S. Cl. 424/401; 514/27; 514/263.31; 514/456

(57) ABSTRACT

The invention relates to the use of the active molecule of formula (I), wherein R═H or R=glucose, for the production of a composition used to activate lipolysis.

The invention relates to a cosmetic composition based on cirsimarin or derivatives of cirsimarin, notably designed for the cosmetic treatment of cellulite.

Adipose tissue is made up of cells called adipocytes, most of whose cellular space is occupied by triglycerides. The quantity of triglycerides contained in an organism depends on both the size and the number of adipocytes. In other terms, adipose cell hypertrophy (increase in their size) and hyperplasia (increase in their number) are important parameters which indicate an increase in the fatty mass. In vivo, hypertrophy and hyperplasia of adipose cells are closely linked mechanisms. It has been proven that an increase in the number of adipocytes is first preceded by an increase in the size of the adipocytes up to a critical threshold, which triggers the recruitment of new adipose cells. Controlling the size parameter alone is enough for treating cellulite cutaneously. Indeed, subcutaneous adipocytes are located in fatty chambers limited by conjunctive tissue. When the size of the adipocytes increases, the fatty chambers are stretched and pressure is applied to the surrounding conjunctive tissue. This pressure causes an orange-peel type wrinkling on the surface of the skin, typical of cellulite. Having observed this, it can be seen that one solution for treating the cellulite phenomenon is to reduce the size of the adipocytes. To do this, it is necessary to catabolize, i.e. to break down, the triglycerides contained in the adipocytes, a phenomenon known as “lipolysis”.

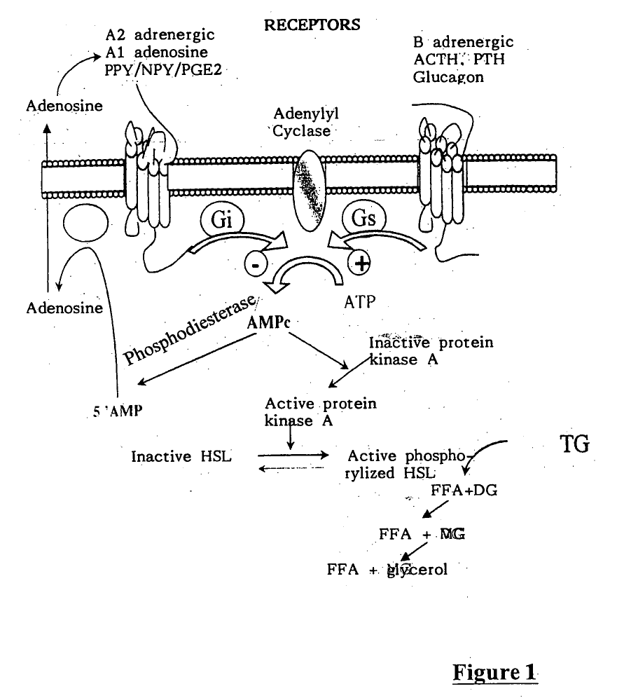

Regulating lipolysis in the adipocytes is a particularly complex phenomenon schematically illustrated in FIG. 1.

The principal lipolytic agents present in the organism are neurotransmitters of the catecholamine type, respectively adrenaline and noradrenaline, neurotransmitters of the sympathetic nervous system.

These catecholamines act on several types of receptors, mainly the β-adrenergic receptors and the α-adrenergic receptors present in the extracellular medium on the surface of the adipocyte membrane.

Adenylate cyclase plays a major role in regulating lipolysis. When it is activated, adenylate cyclase breaks down adenosine triphosphate (ATP) into cyclic adenosine monophosphate (AMPc), which then transforms an inactive protein kinase A into an active phosphorylized protein A, hydrolyzing the triglycerides into di-, and then monoglycerides. In other words, the greater the intracellular AMPc concentration, the more active the lipolysis. This therefore means that, to stimulate lipolysis, it is necessary to increase the intracellular AMPc concentration. This concentration is regulated in several ways.

First of all, AMPc is constantly broken down by phosphodiesterase activated by insulin (an anti-lipolytic agent) into 5′-AMP, which in turn is broken down into adenosine. At the same time, adenosine may migrate from the cell into the extracellular medium, where it attaches to membrane receptors with type A1 adenosine, which themselves are coupled to adenylate cyclase though inhibiting G protein. In other words, adenosine has an inhibiting effect on adenylate cyclase activity, and therefore on lipolysis. This thus means that lipolysis is constantly inhibited and that, when lipolysis is stimulated by stimulating agents, we actually stimulate a system that is constantly inhibited.

Thus, the Applicant's idea is not to stimulate lipolysis directly, but rather to remove the constant inhibition exercised on lipolysis by blocking the A1 adenosine receptors present on the surface of the adipocytes.

Caffeine and theophylline are molecules reported to act as A1 adenosine receptor antagonists, caffeine being known for its lipolytic activity.

The difficulty confronting the Applicant, however, is that not all molecules presenting A1 receptor antagonist properties have sufficient lipolytic activity to be used in cosmetic applications. This is shown in the documents by Nogowski, “Genistein-Induced Changes in Lipid Metabolism of Overiectomized Rats”, Annals of Nutrition and Metabolism, 42(1-2): 360-366 and Jacobson, “Interactions of Flavones and Other Phytochemicals with Adenosine Receptors”, Adv. Exp. Med. Biol., 505: 163-171, in which it is indicated that genistein, a molecule belonging to the flavonoid family, identified as an A1 receptor antagonist, had negligible lipolytic activity.

The problem that the invention proposes to solve is to find an A1 receptor antagonist molecule which has an improved lipolytic effect compared to known molecules, such as caffeine.

Within the context of his research, the Applicant discovered that the molecule with the following formula:

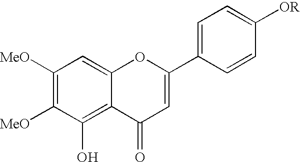

where R=Glucose, or R═H was capable, at very small doses, of removing the constant inhibition exercised on lipolysis.

When the radical, R, is glucose, the molecule used in the invention corresponds to cirsimarin.

Cirsimarin is a flavonoid that belongs to the flavone class. It is a glycolyzed flavone whose raw formula is C₂₃H₂₄O₁₁, corresponding to CAS No. 13020-19-04. It is also known by the following denominations:
5-hydroxy-6,7-dimethoxyflavone 4′-O-glucoside,
5-hydroxyapigenine 6,7-dimethyl ether 4′ glucoside,
Scutellareine 6,7-dimethyl ether 4′-O-glucoside,
Cirsitakaoside,
Cirsimaritin 4′-O-glucoside.

Cirsimarin is found naturally in a very limited number of plants. It can notably be found in Teucrium arduini (Lamiaceae family), Clerodendrum mandarinorum (Verbenaceae family), Scoparia dulcis (Scrophulariacaea family), Cirsium maritimum (Compositae family) and Cirsium pendulum. A more widespread plant in which cirsimarin is found is a creeping grass called Microtea debilis, which belongs to the Phytolaccaceae family, an annual grass from South America. This grass is known for its use in traditional medicine in the form of a dry plant powder administered orally to treat proteinuria.

On this subject, the document “Adenosine-1 Active Ligands: Cirsimarin, a Flavone Glycoside from Microtea debilis”, John A. Hasrat, Luc Pieters, Madga Claeys, Arnold Vlietinck, J. Nat. Prod. 1997, 60, 638-641, points out the A1 receptor antagonist properties of cirsimarin and explains that this molecule's effects on proteinuria is due to the interaction of cirsimarin with the A1 receptors present on the surface of kidney cells. Nothing in this document indicates the possible use of this molecule in a composition designed for activating lipolysis.

Yet the Applicant has demonstrated this property, with cirsimarin's probable mechanism of action corresponding to blockage of the A1 receptors present on the surface of adipocytes.

When the radical, R, is a hydrogenated atom, the molecule used in the invention corresponds to cirsimaritin. This molecule is also known by the following denominations:
Scutellareine 4,7-dimethyl ether,
5,4′-dihydroxy 6,7-dimethoxyflavone,
Cirsistakaogenine,
Scrophuleine,
5-hydroxyapigenine 6,7-dimethyl ether.

Cirsimaritin is found in many crop plants such as Salvia tomentosa (Lamiaceae family), Salvia officinalis, Lippia citriodora (Verbenaceae family), as well as in the wild in Sideretis sventenii (Lamiaceae family), Ocimum gratissimum, gratissimum variety (Lamiaceae family); this list is not limited.

In other words, and in accordance with a first aspect, the invention relates to the use of the molecule with the following formula:

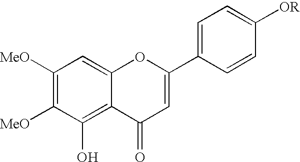

where R=glucose or R═H, for the production of a 10 composition designed to activate lipolysis.

As we have already said, activation of lipolysis would not be caused by the direct stimulation of lipolysis, but rather by relieving the constant inhibition exercised on lipolysis by blocking the A1 receptors present on the surface of adipocytes.

This use may be therapeutic or non-therapeutic.

In the context of this invention, the use is primarily non-therapeutic through localized topical application of cirsimarin or cirsimaritin, in the treatment of cellulite.

The invention thus relates to the use of the aforementioned molecule in a cosmetic composition designed for the topical treatment of cellulite.

The Applicant has also observed that the aforementioned molecule stimulated the synthesis of components of the extracellular matrix. More precisely, it increases the synthesis of structural proteins (collagen, elastin), the synthesis of adherence molecules (collagen, laminin, nidogen, integrin, cadherin, etc.) as well as the synthesis of polysaccharides (glycosaminoglycans and proteoglycans). The invention therefore also relates to the use of the aforementioned molecule in the production of a composition designed for stimulating the synthesis of components of the extracellular matrix.

The invention also includes cosmetic compositions containing, as an active ingredient, cirsimarin or cirsimaritin corresponding to the formulas indicated above.

To be effective, the concentration of cirsimarin or cirsimaritin in the cosmetic composition is between 0.0005% and 10% by weight, advantageously between 0.05% and 5% by weight.

In a special embodiment, cirsimarin is found in the form of a dry or liquid plant extract, preferably of Microtea debilis. When the extract is used in dry form, it represents between 0.005% and 20% and advantageously between 0.1% and 10% by weight in the composition. When the extract is used in liquid form, it represents between 0.1% and 20% and advantageously between 0.5% and 10% by weight in the composition.

In practice, extraction is performed from the whole plant which is dried and then ground, in a polar solvent that can be used in topical cosmetic applications, therefore in an aqueous, alcohol or glycol medium. Generally, the polar solvent is chosen from among the group including water, ethanol, glycols such as propylene glycol, butylene glycol, alone or in a mixture, ethanol, however, being one of the preferred solvent.

The Applicant, furthermore, has demonstrated the existence of synergy between cirsimarin or cirsimaritin and xanthic bases, such as caffeine for example, in lipolysis.

In an advantageous embodiment, the cosmetic composition according to the invention therefore also contains a xanthic base, notably caffeine.

In practice, caffeine represents between 0.1% and 10% by weight of the composition.

Moreover, the composition will generally be formulated in the form of, for example, gel, milk, cream, serum, microemulsion, etc.

The composition according to the invention may be presented in all the galenic forms normally used for topical application on the skin or hair, notably in the form of an aqueous solution, an oil-water, water-oil or multiple emulsion, a silicon emulsion, a microemulsion or nanoemulsion or an aqueous gel.

This composition may be more or less fluid and may take on the appearance, among others, of a white or colored cream, a pomade, a milk, a lotion, a serum or a gel.

The composition according to the invention may contain adjuvants commonly used in the cosmetics and dermatology fields, such as fats, emulsifiers and co-emulsifiers, hydrophilic or lipophylic gelling agents, active hydrophilic or lipophylic ingredients, preservatives, antioxidants, solvents, scents, fillers, hydrophilic or lipophylic filters, colorants, neutralizers, pro-penetrating agents and polymers.

The quantities of the various adjuvants are those conventionally used in the fields in question and, for example, from 0.01% to 30% by total weight of the composition. These adjuvants, depending on their nature, may be included in the oil phase or in the aqueous phase.

The fats that may be used in the invention include mineral oils, oils of animal origin (lanolin), synthetic oils (isopropyl myristate, octyldodecyl, isostearyl isostearate, decyl oleate, isopropyl palmitate), silicon oils (cyclomethicone, dimethicone) and fluorinated oils. The following may be used as fats: fatty alcohols, fatty acids, waxes and gums, notably silicon gums and elastomers.

The emulsifiers and co-emulsifiers that may be used in the invention include, for example, polyglycerol and fatty acid esters, sucrose and fatty acid esters, sorbitan and fatty acid esters, fatty acid and oxyethylene sorbitan esters, fatty alcohol and PEG esters, glycerol and fatty acid esters, alkyl sulfates, alkyl ether sulfates, alkyl phosphates, alkyl polyglucosides and dimethicone copolyols.

The hydrophilic gelling agents that may be used in the invention notably include carboxyvinyl polymers (carbomer), acrylic copolymers such as acrylate/alkylacrylate copolymers, polyacrylamides, polysaccharides such as xanthan gum, guar gum, natural gums such as cellulose gum and derivatives, clays and copolymers of 2-acrylamido-2-methylpropane acid.

The lipophilic gelling agents that may be used in the invention include modified clays such as bentones, metallic salts of fatty acids, hydrophobic silica and ethylcellulose.

The cosmetic composition may also contain active ingredients. These active ingredients may notably include depigmentation agents, emollients, moisturizers, anti-seborrhea agents, anti-acne agents, keratolytic and/or scaling agents, anti-wrinkle and firming agents, draining agents, anti-irritant agents, soothing agents, slimming agents such as xanthic bases (caffeine), vitamins and their mixtures, and matting agents.

In case of incompatibility among them or with the Microtea debilis extract, the active ingredients indicated above and/or the Microtea debilis extract may be encapsulated in spherules, notably ionic or non-ionic vesicles and/or nanoparticles (nanocapsules and/or nanospheres), to isolate them from each other in the composition.

The preservatives that may be used in the invention include benzoic acid, its salts and its esters; sorbic acid and its salts; parabens, their salts and esters; triclosan; imidazolidinyl urea; phenoxyethanol; DMDM hydantoin; diazolidinyl urea and chlorphenesin.

The antioxidants that may be used in the invention include chelating agents such as EDTA and its salts.

The solvents that may be used in the invention include water, ethanol, glycerin, propylene glycol, butylene glycol and sorbitol.

The fillers that may be used in the invention include talc, kaolin, mica, serecite, magnesium carbonate, aluminum silicate, magnesium silicate and organic powders such as nylon.

The filters that may be used in the invention include conventionally used UVA and UVB filters such as benzophenone-3, butyl methoxydibenzoyl methane, octocrylene, octyl methoxycinnamate, 4-methylbenzylidene camphor, octyl salicylate, terephthalylidene dicamphor sulfanic acid and drometrizole trisiloxane. Others can be mentioned such as TiO₂ and ZnO physical filters in their micrometric and nanometric forms.

The colorants that may be used in the invention include lipophylic colorants, hydrophilic colorants, pigments and mother-of-pearl conventionally used in cosmetic or dermatological compositions, and their mixtures.

The neutralizers that may be used in the invention include soda, triethanolamine, aminomethyl propanol and potassium hydroxide.

The pro-penetrating agents that may be used in the invention include alcohols and glycols (ethanol, propylene glycol), ethoxydiglycol, fatty alcohols and acids (oleic acid), fatty acid esters and dimethyl isosorbide.

The composition according to the invention may be used as a care product (for example as a slimming product, as a cleansing product), and/or as a skin makeup product, as a sunscreen product or as a hair care product, for example as a shampoo or conditioner.

The invention also concerns a cosmetic cellulite treatment method, consisting in locally applying an effective quantity of the cosmetic composition in topical application.

The invention and the advantages it provides will be demonstrated in the following embodiment supported by the appended figures.

FIG. 1 is a diagram illustrating the regulation of lipolysis in adipocytes.

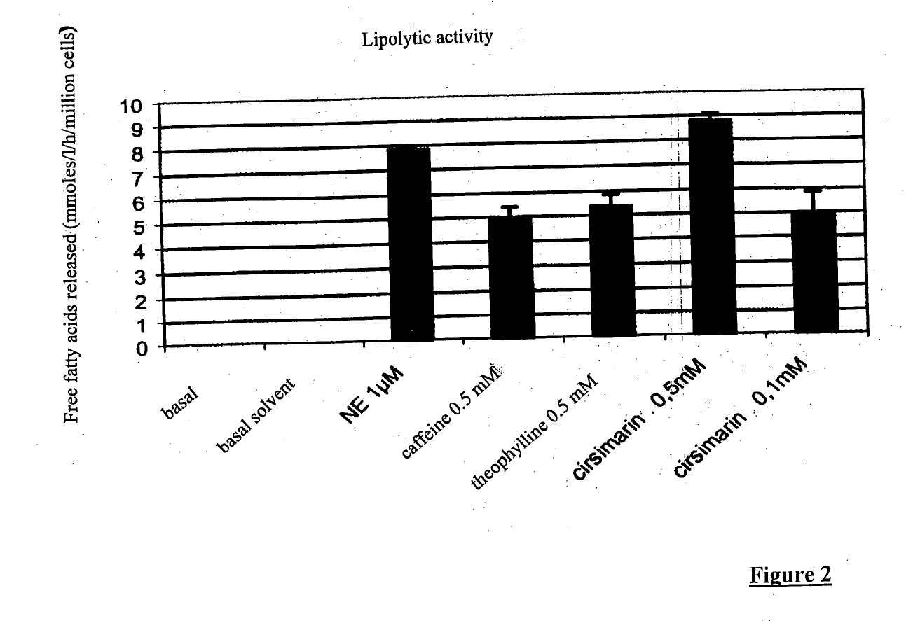

FIG. 2 represents the lipolytic activity of cirsimarin compared to control molecules (caffeine, theophylline, noradrenalin).

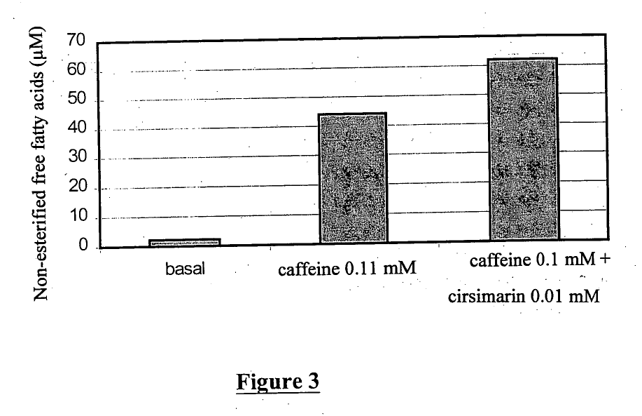

FIG. 3 represents the lipolytic activity of the cirsimarin/caffeine combination compared to caffeine alone.

### EXAMPLE 1

### Production of a Microtea debilis Extract

The whole, dried plant comes from South America. This plant is ground to obtain a powder.

Extraction from the ground plant is performed in a mixture of 96.2% ethanol and H₂O (80/20), volume/volume at room temperature with magnetic shaking, protected from light, for 6 hours.

The extract is then filtered through a nylon filter, then through a cellulose membrane (to 0.22 microns).

### EXAMPLE 2

### Lipolytic Activity of Cirsimarin

This in vitro test on isolated adipocytes demonstrates the lipolytic activity of cirsimarin extracted from Microtea debilis.

2.1/Equipment and Methods

Most of the reagents used come from Sigma-Aldrich.

a. Positive references and products to be assessed
Noradrenalin (syn. Norepinephrine NE): this molecule, with a molar mass of 319.3 g, is assessed at the final concentration of 1 μM in a Krebs-Ringer buffer at 4% albumin.
Caffeine: this molecule, with a molar mass of 194.2 g, is assessed at the final concentration of 0.5 mM in a Krebs-Ringer buffer at 4% albumin.
Theophylline: this molecule, with a molar mass of 180.2, is assessed at the final concentration of 0.5 mM in a Krebs-Ringer buffer at 4% albumin.
Cirsimarin: this molecule, with a molar mass of 476.44, is tested at 0.5 mM and 0.1 mM. This molecule is first solubilized in a mixture of solvents: NaOH 0.2 M/DMSO (95/5; v/v).

b. Reaction Medium

The reactions take place in a Krebs-Ringer bicarbonate buffer containing 4% delipidized albumin (mass/volume), with pH=7.4. The delipidized albumin will fix the fatty acids released during lipolysis; this precaution is important as they retro-inhibit lipolysis. The buffer solution is heated to 37° C.

c. Preparation of the Adipocytes and Start of the Reaction

Epididymal adipose tissue is taken from rats. It is placed in a Krebs-Ringer bicarbonate buffer containing 4% albumin with the pH at 7.4, to which is added 188 units of collagenase/mL to digest the collagen network underlying the adipose tissue.

Digestion takes approximately one hour at 37° C. while shaking. It is stopped by heavily diluting the collagenase with successive rinsing of the adipocyte suspension with Krebs-Ringer buffer containing 4% albumin.

On the Mallasez slide, 8 counts of the adipocyte suspension obtained are performed. The average of these 8 counts is used to distribute the adipocyte solution into reaction tubes at a concentration of approximately 150,000 cells per mL.

The reaction tubes used are 2-mL Eppendorf® tubes. If A is the volume in μL of the adipocyte solution needed to obtain the concentration of 150,000 cells per mL and B is the volume in μL of mother solution of the molecule to be tested, we first place (1,000-A-B) μL of the Krebs-Ringer buffer with 4% albumin in the Eppendorf® tube, followed by B μL of mother solution of the molecule to be tested.

The volume of the tube is then topped up to 1 mL by delicately placing necessary volume of adipocyte solution A on the surface.

All tests are performed 3 times. For the reaction to take place, the tubes are placed in a double-boiler at 37° C. with light lateral shaking. The reaction is stopped after one hour by plunging the tubes into ice.

To determine the basal lipolysis of the adipocytes in the Krebs-Ringer buffer as well as the basal lipolysis of the adipocytes in the Krebs-Ringer buffer containing the cirsimarin solution solvent, control tubes are made. Eight 2-mL Eppendorf® tubes are prepared with A μL of adipocyte solution and (1,000-A) μL of Krebs-Ringer buffer; four tubes are prepared with A μL of the adipocyte solution, B μL of cirsimarin solution solvent and (1,000-A-B) μL of Krebs-Ringer buffer.

Four of the eight tubes containing the Krebs-Ringer buffer and the adipocyte solution are placed directly on ice (tubes 00). The remaining four tubes (tubes 0 Ref) as well as the four tubes containing the Krebs-Ringer buffer, the adipocyte solution and the cirsimarin solution solvent (tubes 0 solvent), are placed in the double boiler and treated in the same way as the reaction tubes.

The lipolytic activity is measured by the quantity of fatty acids released; for this, a NEFA C (Non Esterified Fatty Acid/Colorimetric) colorimetric dosage kit sold by Wako® Chemical GmbH is used.

2.2/Results

The quantity of fatty acids present in tubes 0 minus that present in tubes 00 corresponds to the basal lipolysis of the adipocytes in the buffer. The quantity of fatty acids present in tubes 0 solvent minus that present in tubes 00 corresponds to the basal lipolysis of the adipocytes in the buffer containing the cirsimarin solution solvent. The quantity of fatty acids released in reaction tubes minus that present in tubes 00 corresponds to the lipolysis induced by the molecules studied.

2.3/Conclusions

As this figure shows, cirsimarin has proven in vitro lipolytic activity under the test conditions.

The dilution solvent of Microtea debilis extract has no incidence on basal lipolysis.

### EXAMPLE 3

### Cosmetic Composition

Examples of slimming formulas:

Slimming Body Gel

| Composition | % w/w |
|---|---|
| Carbomer | 0.2 |
| Butylene glycol | 12.0 |
| Phenoxyethanol, methylparaben, | 1.0 |
| butylparaben, ethylparaben, | |
| propylparaben | |
| Sodium hydroxide (10% sol.) | 0.4 |
| Alcohol | 20.0 |
| Ethoxydiglycol | 4.0 |
| Liquid extract of Microtea debilis | 5.0 |
| Glyceryl polymethacrylate and | 10.0 |
| propylene glycol | |
| Water | qs 100.0 |

Slimming Body Milk

| Composition | % w/w |
|---|---|
| PEG-6 stearate and ceteth-20 | 8.0 |
| and steareth-20 | |
| Propylene Glycol Dipelargonate | 10.0 |
| Stearic acid | 1.0 |
| Hydrogenated castor oil | 1.0 |
| Apricot stone oil | 3.0 |
| Dimethicone | 2.0 |
| Tocopheryl acetate | 0.5 |
| Polydecene | 3.0 |
| Cyclomethicone | 3.0 |
| Phenoxyethanol, methylparaben, | 1.0 |
| butylparaben, ethylparaben | |
| and propylparaben | |
| Carbomer | 0.15 |
| Xanthan gum | 0.3 |
| Ethanol | 5.0 |
| Glycerin | 3.0 |
| Sodium hydroxide (10% sol.) | 0.3 |
| Liquid extract of Microtea debilis | 3.0 |
| Ascorbic acid | 0.05 |
| Scent | 0.4 |
| Water | qs 100.0 |

O/W Emulsion

| Composition | Quantity (%) |
|---|---|
| Phenoxyethanol, Methylparaben, | 1 |
| Butylparaben, Ethylparaben, | |
| Propylparaben | |
| Carbomer | 0.4 |
| Glycerin | 3 |
| Xanthan gum | 0.1 |
| Polysorbate-60 | 0.9 |
| Glyceryl Stearate, PEG-100 Stearate | 2.1 |
| Cetyl Alcohol | 2.6 |
| Paraffin oil | 7.5 |
| Isopropyl Myristate | 7.5 |
| Ethoxydiglycol | 5 |
| Dry extract of Microtea debilis | 1 |
| Scent | 0.2 |
| Triethanolamine | 0.3 |
| Water | qs 100 |

W/O Emulsion

| Composition | Quantity (%) |
|---|---|
| Glycerin | 3 |
| Propylene Glycol, Diazolidinyl Urea, | 1 |
| Methylparaben, Propylparaben | |
| Magnesium Sulfate | 0.7 |
| Cetyl Dimethicone Copolyol | 2.5 |
| Isohexadecane | 5 |
| Caprylic/Capric Triglyceride | 5 |
| Dimethicone | 5 |
| Alcohol | 5 |
| Dry extract of Microtea debilis | 2 |
| Scent | 0.1 |
| Water | qs 100 |

Microemulsion

| Composition | Quantity (%) |
|---|---|
| PEG-8 Caprylic/Capric Glycerides | 13.33 |
| Polyglyceryl-6 Dioleate | 8.67 |
| Isostearyl Isostearate | 4 |
| Cyclomethicone | 2.3 |
| Diisopropyl Adipate | 1.6 |
| Octyldodecanol | 2 |
| PPG-5 Ceteth-20 | 2 |
| Phenoxyethanol, Methylparaben, | 0.4 |
| Butylparaben, Ethylparaben, | |
| Propylparaben | |
| Ethoxydiglycol | 2 |
| Dry extract of Microtea debilis | 1 |
| Water | qs 100 |

W/O/W Multiple Emulsion

| Composition | Quantity (%) |
|---|---|
| PEG-30 Dipolyhydroxystearate | 2.4 |
| Isohexadecane | 9 |
| PPG-15 Stearyl Ether | 4.5 |
| Caprylic/Capric Triglyceride | 4.5 |
| Magnesium Sulfate | 0.82 |
| Propylene Glycol, Diazolidinyl Urea, | 1.2 |
| Methylparaben, Propylparaben | |
| Dry extract of Microtea debilis | 2 |
| Poloxamer 407 | 2 |
| Glycerin | 3 |
| Xanthan gum | 0.7 |
| Scent | 0.2 |
| Water | qs 100 |

Sunscreen

| Composition | Quantity (%) |
|---|---|
| DEA Cetyl Phosphate | 2 |
| Glyceryl Stearate, PEG-100 Stearate | 4 |
| Beeswax | 2 |
| Octyl Methoxycinnamate | 7 |
| Butyl Methoxydibenzoylmethane | 2 |
| Benzophenone-3 | 1 |
| Titanium Dioxide | 3 |
| C12/C15 Alkyl Benzoate | 3 |
| Cyclomethicone | 2 |
| Tocopheryl Acetate | 0.5 |
| EDTA | 0.1 |
| Acrylates/C10-30 Alkyl Acrylates | 0.2 |
| Crosspolymer | |
| Xanthan gum | 0.3 |
| Phenoxyethanol, Methylparaben, | 1 |
| Ethylparaben, Propylparaben, | |
| Isobutylparaben | |
| Butylene Glycol | 3 |
| Dry extract of Microtea debilis | 1 |
| Sodium hydroxide (10% sol.) | 0.4 |
| Scent | 0.3 |
| Water | qs 100 |

Makeup Foundation

| Composition | Quantity (%) |
|---|---|
| Glyceryl Stearate, Propylene Glycol | 5 |
| Stearate, Glyceryl Isostearate, | |
| Propylene Glycol Isotearate, | |
| Oleth-25, Ceteth-25 | |
| Glyceryl Dibehenate, Tribehenin, | 1 |
| Glyceryl Behenate | |
| Ethoxydiglycol Oleate | 7.5 |
| Isostearyl isostearate | 5 |
| Cetearyl Alcohol | 2 |
| Dimethicone | 5 |
| Tocopheryl acetate | 0.5 |
| Phenoxyethanol, Methylparaben, Ethylparaben, | 0.6 |
| Propylparaben, Isobutylparaben | |
| Xanthan gum | 0.4 |
| Microcrystalline Cellulose, Cellulose Gum | 1.5 |
| Titanium Dioxide | 6.6 |
| Iron Oxides (Yellow pigment) | 1.55 |
| Iron Oxides (Red pigment) | 0.43 |
| Iron Oxides (Black pigment) | 0.11 |
| Ethoxydiglycol Oleate | 2.5 |
| Dimethicone, Dimethiconol | 3 |
| Alcohol | 5 |
| Dry extract of Microtea debilis | 2 |
| Water | qs 100 |

Shampoo

| Composition | Quantity (%) |
|---|---|
| Acrylates Copolymer | 1.5 |
| Sodium Laurel Sulfate | 5 |
| Sodium Laureth Sulfate | 4 |
| Cocamidopropyl Betaine | 1.5 |
| Polyquaternium-10 | 0.25 |
| DMDM Hydantoin | 0.3 |
| Sodium Hydroxide (20% solution) | 1.3 |
| Citric Acid (50% solution) | 0.7 |
| Dry extract of Microtea debilis | 0.5 |
| Scent | 0.5 |
| Sodium chloride | 0.5 |
| Water | qs 100 |

### EXAMPLE 4

### Synergistic Action of Cirsimarin and Caffeine

In this example, we compare the lipolytic activity of the cirsimarin/caffeine combination to that of caffeine alone on isolated human adipocytes under the same conditions as in example 2. The results are given in FIG. 3.

As this figure shows, there is much greater release of fatty acids with a given quantity (0.11 mM) of the cirsimarin/caffeine combination compared to the same quantity of caffeine (0.11 mM).

1. Use of an active molecule with the following formula: 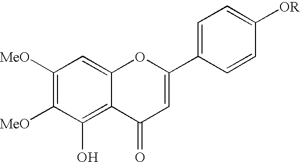
where radical R═H or glucose, for the production of a composition designed to activate lipolysis.

2. Use as claimed in claim 1, wherein when the radical, R, is a glucose molecule, the active molecule corresponds to cirsimarin.

3. Use as claimed in claim 1, wherein when the radical, R, is a hydrogen atom, the molecule corresponds to cirsimaritin.

4. Use as claimed in any one of claims 1 to 3 in a cosmetic composition designed for topical treatment of cellulite.

5. Use as claimed in claim 1 for the production of a composition designed to stimulate synthesis of components of the extracellular matrix.

6. A cosmetic composition containing as its active ingredient a molecule with the following formula: 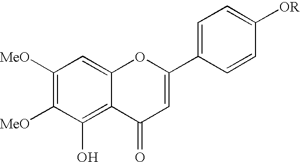
where radical R═H or R=glucose.

7. A composition as claimed in claim 6, wherein when the radical, R, is glucose, the molecule comprises cirsimarin.

8. A composition as claimed in claim 6, wherein when the radical, R, is hydrogen, the molecule comprises cirsimaritin.

9. A cosmetic composition as claimed in claim 6, wherein concentration of the active ingredient is between 0.0005% and 10% by weight.

10. A cosmetic composition as claimed in claim 6, wherein the active ingredient is in the form of a plant extract.

11. A composition as claimed in claim 10, wherein the plant extract comprises an extract of Microtea debilis.

12. A cosmetic composition as claimed in claim 10, wherein when the extract is in dry form, it accounts for between 0.005% and 20% by weight of the composition.

13. A cosmetic composition as claimed in claim 10, wherein when the extract is in liquid form, it accounts for between 0.1% and 20% by weight of the composition.

14. A cosmetic composition as claimed in claim 10, wherein the extract comprises an extract of a whole plant.

15. A cosmetic composition as claimed in claim 6, it also contains further comprising a xanthic base.

16. A cosmetic composition as claimed in claim 15, wherein the xanthic base comprises caffeine.

17. A composition as claimed in claim 16, wherein the caffeine accounts for between 0.1% and 10% by weight of the composition.

18. A cosmetic composition as claimed in claim 6, further comprising a cosmetically acceptable vehicle.

19. A cellulite treatment method comprising locally applying an effective quantity of the cosmetic composition of any one of claims 6 to 18, in topical application.
